(19) United States
(12) Patent Application Publication
(10) Pub. No.: US 2002/0119442 A1
(43) Pub. Date: Aug. 29, 2002

(54) Approaches to identify genetic traits

(76) Inventors: Charles L.M. Dunlop, Irvine, CA (US); James M. Weisel, Manhattan Beach, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/851,501

(22) Filed: May 8, 2001

Related U.S. Application Data
(63) Continuation-in-part of application No. PCT/US00/30493, filed on Nov. 3, 2000.
(60) Provisional application No. 60/165,301, filed on Nov. 12, 1999.

(51) Int. Cl. C12Q 1/70; C12Q 1/68; C12P 19/34

(52) U.S. Cl. 435/5; 435/6; 435/91.2

(57) ABSTRACT

The present invention relates to the field of genetic screening. More specifically, the described embodiments concern methods to screen multiple samples, in a single assay, for the presence or absence of mutations or polymorphisms in a plurality of genes.

### CROSS-REFERENCE TO RELATED APPLICATIONS

This application is a continuation-in-part of International Application number PCTUS00/30493, filed Nov. 3, 2000, and claims priority to said International Application and U.S. Provisional Patent Application No. 60/165,301, filed Nov. 12, 1999. Both International Application number PCTUS00/30493 and U.S. Provisional Patent Application No. 60/165,301 are hereby expressly incorporated by reference in their entireties.

### FIELD OF THE INVENTION

The present invention relates to the field of genetic screening. More specifically, the described embodiments concern methods to screen multiple samples, in a single assay, for the presence or absence of mutations or polymorphisms in a plurality of genes.

### BACKGROUND OF THE INVENTION

Despite the tremendous progress in molecular biology and the identification of genes, mutations, and polymorphisms responsible for disease, the ability to rapidly screen a subject for the presence of multiple disorders has been technically difficult and cost prohibitive. Current DNA-based diagnostics allow for the identification of a single mutation or polymorphism or gene per analysis. Although high-throughput methods and gene chip technology have enabled the ability to screen multiple samples or multiple loci within the same sample, these approaches require several independent reactions, which increases the time required to process clinical samples and drastically increases the cost. Further, because of time and expense, conventional diagnostic approaches focus on the identification of the presence of DNA fragments that are associated with a high frequency of mutation, leaving out analysis of other loci that may be critical to diagnose a disease. The need for a better way to diagnose genetic disease is manifest.

With the advent of multiplex Polymerase Chain Reaction (PCR), the ability to use multiple primer sets to generate multiple extension products from a single gene is at hand. By hybridizing isolated DNA with multiple sets of primers that flank loci of interest on a single gene, it is possible to generate a plurality of extension products in a single PCR reaction corresponding to fragments of the gene. As the number of primers increases, however, the complexity of the reaction increases and the ability to resolve the extension products using conventional techniques fails. Further, since many diseases are caused by changes of a single nucleotide, the rapid detection of the presence or absence of these mutations or polymorphisms is frustrated by the fact that the PCR products that indicate both the diseased and non-diseased state are of the same size.

Developments in gel electrophoresis and high performance liquid chromatography (HPLC), however, have enabled the separation of double-stranded DNAs based upon differences in their melting behaviors, which has allowed investigators to resolve DNA fragments having a single mutation or single polymorphism. Techniques such as temporal temperature gradient gel electrophoresis (TTGE) and denaturing high performance liquid chromatography (DHPLC) have been used to screen for small changes or point mutations in DNA fragments.

The separation principle of TTGE, for example, is based on the melting behavior of DNA molecules. In a denaturing polyacrylamide gel, double-stranded DNA is subject to conditions that will cause it to melt in discrete segments called “melting domains.” The melting temperature Tₘ of these domains is sequence-specific. When the Tₘ of the lowest melting domain is reached, the DNA will become partially melted, creating branched molecules. Partial melting of the DNA reduces its mobility in a polyacrylamide gel.

Since the Tₘ of a particular melting domain is sequence-specific, the presence of a mutation or polymorphism will alter the melting profile of that DNA in comparison to the wild-type or non-polymorphic DNA. That is, a heteroduplex DNA consisting of a wild-type or non-polymorphic strand annealed to mutant or poymorphic strand, will melt at a lower temperature than a homoduplex DNA strand consisting of two wild-type or non-polymorphic strands. Accordingly, the DNA containing the mutation or polymorphism will have a different mobility compared to the wild-type or non-polymorphic DNA. The TTGE approach has been used as a method for screening for mutations in the cystic fibrosis gene, for example. (Bio-Rad U.S./E.G. Bulletin 2103, herein expressly incorporated by reference in its entirety).

Similarly, the separation principle of DHPLC is based on the melting or denaturing behavior of DNA molecules. As the use and understanding of HPLC developed, it became apparent that when HPLC analyses were carried out at a partially denaturing temperature, i.e., a temperature sufficient to denature a heteroduplex at the site of base pair mismatch, homoduplexes could be separated from heteroduplexes having the same base pair length. (See e.g., Hayward-Lester, et al., Genome Research 5:494 (1995); Underhill, et al., Proc. Natl. Acad. Sci. USA 93:193 (1996); Oefner, et al., DHPLC Workshop, Stanford University, Palo Alto, Calif., (Mar. 17, 1997); Underhill, et al., Genome Research 7:996 (1997); Liu, et al., Nucleic Acid Res., 26:1396 (1998), all of which and the references contained therein are hereby expressly incorporated by reference in their entireties). Techniques such as Matched Ion Polynucleotide Chromatography (MIPC) and Denaturing Matched Ion Polynucleotide Chromatography (DMIPC) have also been employed to increase the sensitivity of detection. It was soon realized that DHPLC, which for the purposes of this disclosure includes but is not limited to, MIPC, DMIPC, and ion-pair reverse phase high-performance liquid chromatography, could be used to separate heteroduplexes from homoduplexes that differed by as little as one base pair. Various DHPLC techniques have been described in U.S. Pat. Nos. 5,795,976; 5,585,236; 6,024,878; 6,210,885; Huber, et al., Chromatographia 37:653 (1993); Huber, et al., Anal. Biochem. 212:351 (1993); Huber, et al., Anal. Chem. 67:578 (1995); O'Donovan et al., Genomics 52:44 (1998), Am J Hum Genet. December;67(6):1428-36 (2000); Ann Hum Genet. September:63 (Pt 5):383-91 (1999); Biotechniques, April;28(4):740-5 (2000); Biotechniques. November;29(5):1084-90, 1092 (2000); Clin Chem. August;45(8 Pt 1):1133-40 (1999); Clin Chem. April;47(4):635-44 (2001); Genomics. August 15;52(1):44-9 (1998); Genomics. March 15;56(3):247-53 (1999); Genet Test.;1(4):237-42 (1997-98); Genet Test.:4(2):125-9 (2000); Hum Genet. June;106(6):663-8 (2000); Hum Genet. November;107(5):483-7 (2000); Hum Genet. November;107(5):488-93 (2000); Hum Mutat. December;16(6):518-26 (2000); Hum Mutat. 15(6):556-64 (2000); Hum Mutat. March;17(3):210-9 (2001); J Biochem Biophys Methods. November 20;46(1-2):83-93 (2000); J Biochem Biophys Methods. January 30;47(1-2):5-19 (2001); Mutat Res. November 29;430(1):13-21(1999); Nucleic Acids Res. March 1;28(5):E13 (2000); and Nucleic Acids Res. October 15;28(20):E89 (2000), all of which, including the references contained therein, are hereby expressly incorporated by reference in their entireties. Despite the efforts of many, there remains a need for a better approach to screen for mutations and/or polymorphisms.

### BRIEF SUMMARY OF THE INVENTION

Embodiments described herein concern a novel approach to screen for the presence or absence of multiple mutations or polymorphisms in a plurality of genes in a single assay, thus, improving the speed and lowering the cost to diagnose genetic diseases. Several embodiments also permit very sensitive detection of single base mutations, single base mismatches, and small nuclear polymorphisms (SNPs), as well as, larger alterations in DNA at multiple loci, in a plurality of genes, in multiple samples. Further, by employing a DNA standard or by screening a plurality of DNA samples in the same assay, improved sensitivity of detection can be obtained.

Embodiments include a method of identifying the presence or absence of a plurality of genetic markers in a subject. One method is practiced, for example, by providing a DNA sample from said subject, providing a plurality of nucleic acid primer sets that hybridize to said DNA at regions that flank said plurality of genetic markers, wherein each primer set has a first and a second primer and, wherein said plurality of genetic markers exist on a plurality of genes, contacting said DNA and said plurality of nucleic acid primer sets in a single reaction vessel, generating, in said single reaction vessel, a plurality of extension products that comprise regions of DNA that include the location of said plurality of genetic markers, separating said plurality of extension products on the basis of melting behavior, and identifying the presence or absence of said plurality of genetic markers in said subject by analyzing the melting behavior of said plurality of extension products. In some aspects of this method the separation on the basis of melting behavior is accomplished by TTGE and in other embodiments the separation on the basis of melting behavior is accomplished by DHPLC. In some embodiments, said extension products are first separated by size for a period sufficient to separate populations of extension products and then separated by melting behavior. The size separation can be accomplished on the TTGE gel or DHPLC column prior to separating on the basis of melting behavior.

In some embodiments, the subject is selected from the group consisting of a plant, virus, bacteria, mold, yeast, animal, and human and either the first or the second primer comprise a GC clamp. In other aspects of this embodiment, either the first or the second primer hybridize to a sequence within an intron. Preferably, at least one of the plurality of genetic markers is indicative of a disease selected from the group consisting of familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis coleceral cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease.

In other embodiments, the plurality of primer sets consist of at least 3, 4, 5, 6, or 7 primer sets. Additionally, in some embodiments, the plurality of genes consist of at least 2, 3, 4, 5, 6, or 7 genes. The method above preferably generates the extension products using the Polymerase Chain Reaction and the method can be supplemented by a step in which a control DNA is added.

Another embodiment concerns a method of identifying the presence or absence of a plurality of genetic markers in a plurality of subjects. This method is practiced by: providing a DNA sample from said plurality of subjects, providing a plurality of nucleic acid primer sets that hybridize to said DNA at regions that flank said plurality of genetic markers, wherein each primer set has a first and a second primer and, wherein said plurality of genetic markers exist on a plurality of genes, contacting said DNA and said plurality of nucleic acid primer sets in a single reaction vessel, generating, in said single reaction vessel, a plurality of extension products that comprise regions of DNA that include the location of said plurality of genetic markers, separating said plurality of extension products on the basis of melting behavior, and identifying the presence or absence of said plurality of genetic markers in said plurality of subjects by analyzing the melting behavior of said plurality of extension products. In some aspects of this embodiment, the separation on the basis of melting behavior is accomplished by TTGE and in other embodiments the separation on the basis of melting behavior is accomplished by DHPLC.

In other embodiments, the subject is selected from the group consisting of a plant, virus, bacteria, mold, yeast, animal, and human and either the first or the second primer comprise a GC clamp. In other aspects of this embodiment, either the first or the second primer hybridize to a sequence within an intron. Preferably, at least one of the plurality of genetic markers is indicative of a disease selected from the group consisting of familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis coleceral cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease.

In more embodiments, the plurality of subjects consist of at least 2, 3, 4, 5, 6, or 7 subjects. In more aspects of this embodiment, the plurality of primer sets consist of at least 3, 4, 5, 6, or 7 primer sets. Additionally, in some embodiments, the plurality of genes consist of at least 2, 3, 4, 5, 6, or 7 genes. The method above preferably generates the extension products using the Polymerase Chain Reaction and the method can be supplemented by a step in which a control DNA is added.

Still another embodiment involves a method of identifying the presence or absence of a mutation or polymorphism in a subject. This method is practiced by: providing a DNA sample from said subject, generating a population of extension products from said sample, wherein said extension products comprise a region of said DNA that corresponds to the location of said mutation or polymorphism, providing at least one control DNA, wherein said control DNA lacks said mutation or polymorphism, contacting said control DNA and said population of extension products in a single reaction vessel thereby forming a mixed DNA sample, heating said mixed DNA sample to a temperature sufficient to denature said control DNA and said DNA sample, cooling said mixed DNA sample to a temperature sufficient to anneal said control DNA and said DNA sample, separating said mixed DNA sample on the basis of melting behavior, and identifying the presence or absence of said mutation or polymorphism by analyzing the melting behavior of said mixed DNA sample. In some aspects of this embodiment, the control DNA is DNA obtained from a second subject and the presence or absence of a mutation or polymorphism is not known. In some aspects of this embodiment, the separation on the basis of melting behavior is accomplished by TTGE and in other embodiments the separation on the basis of melting behavior is accomplished by DHPLC.

Still more embodiments concern isolated or purified nucleic acids consisting of a sequence selected from the group consisting of SEQ. ID. Nos. 1-44 and kits containing said nucleic acids. These nucleic acid primers can be used to efficiently determine the presence or absence of a polymorphism or mutation in a multiplex PCR reaction that screens a plurality of genes and a plurality of subjects in a single reaction vessel. Additionally, reaction vessels comprising a DNA sample, and a plurality of nucleic acid primer sets (e.g., SEQ. ID. Nos. 1-44) that hybridize to said DNA sample at regions that flank a plurality of genetic markers, wherein said plurality of genetic markers exist on a plurality of genes are embodiments. Further, a reaction vessel comprising a plurality of DNA samples obtained from a plurality of subjects and a plurality of nucleic acid primer sets (e.g., SEQ. ID. Nos. 1-44) that hybridize to said plurality of DNA samples at regions that flank a plurality of genetic markers, wherein said plurality of genetic markers exist on a plurality of genes.

Other embodiments concern a gel having lanes and adapted to separate different DNAs comprising a plurality of extension products, in a single lane of said gel, wherein said plurality of extension products correspond to regions of DNA located on a plurality of genes and, wherein said regions of DNA comprise loci that indicate a genetic trait and a gel having lanes and adapted to separate different DNAs comprising a plurality of extension products, in a single lane of said gel, wherein said plurality of extension products correspond to regions of DNA located on a plurality of genes in a plurality of subjects and, wherein said regions of DNA comprise loci that indicate a genetic trait.

Additional embodiments include a DHPLC column adapted to separate different DNAs comprising a plurality of extension products, wherein said plurality of extension products correspond to regions of DNA located on a plurality of genes and, wherein said regions of DNA comprise loci that indicate a genetic trait and a DHPLC column adapted to separate different DNAs comprising a plurality of extension products, wherein said plurality of extension products correspond to regions of DNA located on a plurality of genes in a plurality of subjects and, wherein said regions of DNA comprise loci that indicate a genetic trait.

### DETAILED DESCRIPTION OF THE INVENTION

The invention described herein concerns approaches to analyze DNA samples for the presence or absence of a plurality of genetic markers that reside on a plurality of genes in a single assay. Some embodiments allow one to rapidly distinguish a plurality of DNA fragments in a single sample that differ only slightly in size and/or composition (e.g., a single base change, mutation, or polymorphism). Other embodiments concern methods to screen multiple genes from a subject, in a single assay, for the presence or absence of a mutation or polymorphism. An approach to achieve greater sensitivity of detection of mutations or polymorphisms present in a DNA sample is also provided. Preferred embodiments, however, include methods to screen multiple genes, in a plurality of DNA samples, in a single assay, for the presence or absence of mutations or polymorphisms.

It was discovered that multiple extension products that have slight differences in length and/or composition can be resolved by separating the DNA on the basis of melting temperature. By one approach, a plurality of varying lengths of double-stranded DNA are applied to a denaturing gel and the double-stranded DNAs are separated by applying an electrical current while the temperature of the gel is raised gradually. By slowly increasing the temperature while the DNA is electrically separated on a polyacrylamide gel containing a denaturant (e.g., urea), the dsDNA eventually denatures to partially single stranded (branched molecules) DNA. Because branched or heteroduplex DNA migrates more rapidly or more slowly than dsDNA or homoduplex DNA, one can quickly determine the differences in melting behavior between DNA fragments, compare this melting temperature to a standard DNA (e.g., a wild-type DNA or non-polymorphic DNA), and identify the presence or absence of a mutation or polymorphism in the screened DNA. This technique efficiently separates multiple DNA fragments, generated by a single multiplex PCR reaction on a plurality of loci from different genes (e.g., in one experiment, 10 different loci were analyzed in the same reaction and each of the extension products, some that differed by only a single mutation, were efficiently resolved).

It was also discovered that multiple extension products that have slight differences in length and/or composition can be resolved by separating the DNA by DHPLC. By one approach, a plurality of varying lengths of double-stranded DNA are applied to a ion-pair reverse phase HPLC column (e.g., alkylated non-porous poly(styrene-divinylbenzene)) that has been equilibrated to an appropriate denaturing temperature, depending on the size and composition of the DNA to be separated (e.g., 53° C. to 63° C.) in an appropriate buffer (e.g., 0.1 mM triethylamine acetate (TEAA) pH 7.0). Once applied to the column, the double stranded DNA binds to the matrix. By slowly increasing the presence of a denaturant (e.g., acetonitrile in TEAA), the dsDNA eventually denatures to partially single stranded (branched molecules) DNA and elutes from the column. Preferably a linear gradient is used to slowly elute the bound DNA. Detection can be accomplished using a U.V. detector, radioactivity, dyes, or fluoresence. In some embodiments, the extension products are first separated on the basis of size using a shallow gradient of denaturant for a time sufficient to separate individual populations of extension products and then on the basis of melting behavior using a deeper gradient of denaturant. The techniques described in the following references can also be modified for use with aspects of the invention: U.S. Pat. Nos. 5,795,976; 5,585,236; 6,024,878; 6,210,885; Huber, et al., Chromatographia 37:653 (1993); Huber, et al., Anal. Biochem. 212:351 (1993); Huber, et al., Anal. Chem. 67:578 (1995); O'Donovan et al., Genomics 52:44 (1998), Am J Hum Genet. December;67(6):1428-36 (2000); Ann Hum Genet. September:63 (Pt 5):383-91 (1999); Biotechniques, April;28(4):740-5 (2000); Biotechniques. November;29(5):1084-90, 1092 (2000); Clin Chem. August;45(8 Pt 1): 1133-40 (1999); Clin Chem. April;47(4):635-44 (2001); Genomics. August 15;52(1):44-9 (1998); Genomics. March 15;56(3):247-53 (1999); Genet Test.;1(4):237-42 (1997-98); Genet Test.:4(2):125-9 (2000); Hum Genet. June;106(6):663-8 (2000); Hum Genet. November;107(5):483-7 (2000); Hum Genet. November;107(5):488-93 (2000); Hum Mutat. December;16(6):518-26 (2000); Hum Mutat. 15(6):556-64 (2000); Hum Mutat. March;17(3):210-9 (2001); J Biochem Biophys Methods. November 20;46(1-2):83-93 (2000); J Biochem Biophys Methods. January 30;47(1-2):5-19 (2001); Mutat Res. November 29;430(1):13-21(1999); Nucleic Acids Res. March 1;28(5):E13 (2000); and Nucleic Acids Res. October 15;28(20):E89 (2000), all of which are hereby expressly incorporated by reference in their entireties including the references cited therein.

Because branched or heteroduplex DNA elutes either more rapidly or more slowly than homoduplex DNA, one can quickly determine the differences in melting behavior between DNA fragments, compare this melting temperature to a standard DNA (e.g., a wild-type or non-polymorphic homoduplex DNA), and identify the presence or absence of a mutation or polymorphism in the screened DNA. This technique efficiently separates multiple DNA fragments, generated by a single multiplex PCR reaction on a plurality of loci from different genes.

Some of the embodiments described herein have adapted the DNA separation techniques described above to allow for high-throughput genetic screening of organisms (e.g., plant, virus, bacteria, mold, yeast, and animals including humans). Typically, multiple primers that flank genetic markers (e.g., mutations or polymorphisms that indicate a congenital disease or a trait) on different genes are employed in a single amplification reaction and the multiple extension products are separated on a denaturing gel or by DHPLC according to their melting behavior. The presence or absence of mutations or polymorphisms, also referred to as “genetic markers”, in the subject's DNA are then detected by identifying an aberrant melting behavior in the extension products (e.g., migration on a gel that is too fast or too slow or elution from a DHPLC column that is too fast or too slow). Advantageously, some embodiments provide a greater understanding of a subject's health because more loci that are indicative of disease, for example, are analyzed in a single assay. Further, some embodiments drastically reduce the cost of performing such diagnostic assays because many different genes and markers for disease can be screened simultaneously in a single assay.

By one approach, for example, a biological sample from the subject (e.g., blood) is obtained by conventional means and the DNA is isolated. Next, the DNA is hybridized with a plurality of nucleic acid primers that flank regions of a plurality of genetic loci or markers that are associated with or linked to the plurality of traits to be analyzed. Although different loci have been detected in a single assay (requiring 20 primers), more or less loci can be screened in a single assay depending on the needs of the user. Preferably, each assay has sufficient primers to screen at least three different loci, which may be located on three different genes. That is, the embodied assays can have sufficient primers to screen at least 3, 4, 5, 6, 7, 8, 9, 10, 11, 12, 13, 14, 15, 16, 17, 18, 19, 20, or more, independent loci or markers that are indicative of a disease in a single assay and these loci can be on different genes. Because more than one loci or marker can be detected by a single set of primers, the detection of 20 different markers, for example, can be accomplished with less than 40 primers. However, in many assays, a different set of primers is needed to detect each different loci. Thus, in several embodiments, at least 6, 8, 10, 12, 14, 16, 18, 20, 22, 24, 26, 28, 30, 32, 34, 36, 38, 40, or more primers are used.

Desirably, the primers hybridize to regions of human DNA that flank markers or loci associated with or linked to human diseases such as: familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis coleceral cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease. It should be understood, however, that the list above is not intended to limit the invention in any way and the techniques described herein can be used to detect and identify any gene or mutation or polymorphism desired (e.g., polymorphisms or mutations associated with alcohol dependence, obesity, and cancer).

Once the primers are hybridized to the subject's DNA, a plurality of extension products having the marker or loci indicative of the trait are generated. Preferably, the extension products are generated through a polymerase-driven amplification reaction, such as multiplex PCR or multiplex Ligase Chain Reaction (LCR). Then the extension products are separated on the basis of melting behavior (e.g., TTGE or DHPLC).

In some approaches, for example, the extension products are isolated from the reactants in the amplification reaction, suspended in a non-denaturing loading buffer, and are loaded on a TTGE denaturing gel (e.g., an 8%, 7M urea polyacrylamide gel). The sample can be heated to a temperature sufficient to denature a DNA duplex and then cooled to a temperature that allows reannealing, prior to suspending the DNA in the non-denaturing loading buffer. The extension products are then loaded into a single lane or multiple lanes, as desired. Next, an electrical current is applied to the gel and extension products.

Subsequently, the temperature of the denaturing gel is gradually raised, while maintaining the electrical current, so as to separate the extension products on the basis of their melting behaviors. Once the fragments have been separated by size and melting behavior, one can identify the presence or absence of mutations or polymorphisms at the screened loci by analyzing the migration behavior of the extension products.

In other approaches, the extension products are isolated from the reactants and suspended in a DHPLC buffer (e.g., 0.1M TEAA pH 7.0). The extension products are then injected onto a DHPLC column (e.g., an ion-pair reverse phase HPLC column composed of alkylated non-porous poly(styrene-divinylbenzene)) that has been equilibrated to an appropriate denaturing temperature, depending on the size and composition of the DNA to be separated (e.g., 53° C. to 63° C.) in an appropriate buffer (e.g., 0.1 mM triethylamine acetate (TEAA) pH 7.0) and the extension products are allowed to bind. The presence of a denaturant (e.g., acetonitrile in TEAA) on the column is gradually raised over time so as to slowly elute the extension products from the column. Preferably a linear gradient is used. Presence of the extension products in the eluant is preferably accomplished using a UV detector (e.g., at 260 and/or 280 nm), however, greater sensitivity may be obtained using radioactivity, binding dyes, fluorescence or the techniques described in U.S. Pat. Nos. 5,795,976; 5,585,236; 6,024,878; 6,210,885; Huber, et al., Chromatographia 37:653

Extension products are then generated. If the subject being tested has at least one trait that is detected by the assay (e.g., a congenital disorder), then two populations of extension products are generated, a first population that corresponds to the standard DNA and a second population that corresponds to the subject's DNA having at least one mutation or polymorphism. Next, preferably, the two populations of extension products are isolated from the amplification reactants and are denatured by heat (e.g., 95° C. for 5 minutes), then are allowed to anneal by cooling (e.g., ice for 5 minutes). This ensures the formation of the heteroduplex bands in the presence of any relatively small mutation (e.g., point mutation, small insertion, or small deletion). The isolation and denaturing/annealing steps are not practiced with some embodiments, however.

Subsequently, by the TTGE approach, the two populations of extension products are suspended in a non-denaturing loading buffer and loaded on a denaturing polyacrylamide gel and separated on the basis of melting behavior, as described above. By the DHPLC approach, the two populations of extension products are suspended in a suitable buffer (e.g., 0.1M TEAA pH 7.0), loaded onto a buffer and temperature equilibrated DHPLC column and a linear gradient of denaturant is applied, as described above. Because the two populations of extension products are not perfectly complementary, they form heteroduplexes. Heteroduplexes are less stable than homoduplexes, have a lower melting temperature, and are easily differentiated from homoduplexes using the DNA separation techniques described above. One can identify the presence or absence of mutations or polymorphisms at the screened loci, for example, by comparing the migration behavior or elution behavior of the extension products generated from the screened DNA with the migration behavior or elution behavior of the DNA standard. If heteroduplexes are present, generally, two additional bands that correspond to the single extension product will appear on the gel or the extension products will elute from the column more rapidly than the control or standard DNA alerting the user to the presence of a mutation or polymorphism. Accordingly, a significant increase in sensitivity is obtained and a user can rapidly identify the presence or absence of a mutation or polymorphism in the tested DNA sample and, thereby, determine whether the screened subject has a predilection for a particular trait (e.g., a congenital disease).

Similarly, an increase in sensitivity can be obtained by mixing DNA from a plurality of subjects prior to amplification. Because the frequency of mutations or polymorphisms for most disorders are very low in the population, most of the extension products generated are wild-type DNA. Thus, most of the pool of DNA behaves as a DNA standard. That is, the predominant structure formed upon annealing after denaturation is a homoduplex, which can be rapidly distinguished from any heteroduplex that would appear if a subject were to have a polymorphism or mutation. Of course, extension products previously generated from multiple subjects can be used as control DNA by mixing the previously generated extension products with the extension products generated from the DNA that is being screened prior to electrophoresis. In several embodiments, the DNA from at least 2 subjects is mixed. Desirably, the DNA from at least 3 subjects is mixed. Preferably, the DNA from at least 4 subjects is mixed. It should be understood, however, that the DNA from at least 2, 3, 4, 5, 6, 7, 8, 9, 10, 11, 12, 13, 14, 15, 16, 17, 18, 19, 20, or more subjects can be mixed prior to amplification or prior to separation on the basis of melting behavior, in accordance with some of the described embodiments.

In one embodiment, for example, DNA from a plurality of subjects to be tested is obtained by conventional methods, pooled, and hybridized with the desired nucleic acid primers. Extension products are then generated, as before. If at least one of the subjects being tested has at least one congenital disorder that is detected by the screen then two populations of extension products will be generated, a first population that corresponds to DNA from subjects that have the wild-type gene and a second population that corresponds to DNA from subjects having at least one mutant or polymorphic gene.

By one approach, the two populations of extension products are then isolated from the amplification reactants, suspended in a non-denaturing loading buffer, denatured by heat, annealed by cooling, and are separated by TTGE, as described above. By another approach, the two populations of extension products are isolated from the amplification reactants, suspended in a DHPLC loading buffer (0.1M TEAA pH 7.0), denatured by heat, annealed by cooling, and are separated on a DHPLC column, as described above. The presence of a subject in the DNA pool having at least one mutation or polymorphism is identified by analyzing the migration behavior of the DNA on the gel or the elution behavior from the column. The appearance of a slower or faster migrating band at a temperature below or above the predicted melting point for a particular extension product on the gel indicates the presence of a mutation or polymorphism in the DNA from one of the subjects. Similarly, the appearance of a slower or faster eluting extension product from the DHPLC column indicates the presence of a mutation or polymorphism in the DNA from one of the subjects. By repeating the analysis with smaller and smaller pools of samples, one can identify the individual(s) in the pool that has the mutation or polymorphism. Additionally, DNA standards can be used, as described above, to facilitate identification of the individual(s) having the mutation or polymorphism. Advantageously, some embodiments can be used to screen multiple samples at multiple loci that are on found on a plurality of genes in a single assay, thus, increasing sample throughput. The analysis of a plurality of DNA samples in the same assay also unexpectedly provides greater sensitivity. The section below describes a DNA separation technique that can be used with the embodiments described herein.

Multiple Extension Products of Similar Composition can be Separated on the Same Lane of a Denaturing Gel or in the Same Run on a DHPLC Column

It was discovered that multiple fragments of DNA, which vary slightly in length and/or composition, can be rapidly and efficiently resolved on the basis of melting behavior. Although the preferred methods for differentiating multiple fragments of DNA on the basis of melting behavior involve TTGE gel electrophoresis and DHPLC, it is contemplated that other conventional techniques that are amenable to DNA separation on the basis of melting behavior can be equivalently employed (e.g., size exclusion chromatography, ion exchange chromatography, and reverse phase chromatography on high pressure (e.g., HPLC), low pressure (e.g., FPLC), gravity-flow, or spin-columns, as well as, thin layer chromatography).

By one approach, a polyacrylamide gel having a porosity sufficient to resolve the DNA fragments on the basis of size (e.g., 4-20% acrylamide/bis acrylamide gel having a set concentration of denaturant) is used. The amount of denaturant in the gel (e.g., urea or formamide) can vary according to the length and composition of the DNA to be resolved. The concentration of urea in a polyacrylamide gel, for example, can be 3M, 3.5M, 4M, 4.5M, 5M, 5.5M, 6M, 6.5M, 7M, 7.5M, or 8M. In preferred embodiments, an 8% is wild-type or non-polymorphic for all of the loci that are being screened. Because this DNA separation technique is sufficiently sensitive to identify a single base pair substitution in a DNA fragment up to 600 base pairs in length, small changes in the melting behaviors and migration of the extension products can be rapidly identified.

By another approach, DHPLC is used to resolve heteroduplex and homoduplex molecules of several PCR extension products in a single assay. Preferably, the heteroduplex and homoduplex extension products are separated from each other by ion-pair reverse phase high performance liquid chromatography. In one embodiment, a DHPLC column that contains alkylated non-porous poly(styrene-divinylbenzene) is used. Preferably, the DHPLC column is equilibrated in an appropriate degassed buffer, referred to as Buffer “A” (e.g., 0.1M TEAA pH 7.0) and is kept at a constant temperature somewhat below the predicted melting temperature of the extension products (e.g., 53° C.-60° C., preferably 50° C.). A plurality of extension products that may be generated from a plurality of different loci, as described herein, are suspended in Buffer A and are injected onto the DHPLC column. The Buffer A is then allowed to run through the column for a time sufficient to insure that the extension products have adequately bound to the column. Preferably, flow rate and the amount of gas (e.g., argon or helium) are adjusted and kept constant so that the pressure on the column does not exceed the recommended level. Gradually, degassed denaturing buffer, referred to as Buffer “B”, (e.g., 0.1M TEAA pH 7.0 and 25% acetonitrile) is applied to the column. Although an isocratic gradient can be used, a gradual linear gradient is preferred. By one approach, to separate fragments that range in size from 200-450 bp, for example, a gradient of 50%-65% Buffer B (0.1M TEAA pH 7.0 and 25% acetonitrile) is used. Of course, as the size of extension products to be separated on the DHPLC column decreases, the gradient and/or the amount of denaturant in Buffer B can be reduced, whereas, as the size of extension products to be separated on the DHPLC column increases, the gradient and/or the amount of denaturant in Buffer B can be increased.

The DHPLC column is designed such that double stranded DNA binds well but as the extension products become partially denatured the affinity to the column is reduced until a point is reached at which the particular extension product can no longer adhere to the column matrix. Typically, heteroduplexes denature before homoduplexes, thus, they would be expected to elute more rapidly from the column than homoduplexes.

In some embodiments, particularly embodiments concerning the separation of a plurality of different extension products (e.g., extension products generated from a plurality of loci), the choice of primers and, thus, the extension products generated therefrom, requires careful design. For exanple, a GC-clamp or other artificial sequence can be used to adjust the melting characteristics and increase the length of a particular DNA fragment, if needed, to facilitate separation on the DHPLC or improve resolution of the extension products. By one approach, each set of primers in a multiplex reaction are designed and selected to generate an extension product that has a unique homoduplex and heteroduplex elution behavior. In this manner, each species can be easily identified.

By another approach, each set of primers are designed to generate extension products that have homoduplexes with very similar melting characteristics. By this strategy, all of the homoduplexes will elute at the same or very similar concentration of denaturant, which is different than the concentration of denaturant required to elute the heteroduplexes. Accordingly, the elution of a species of extension product outside of the expected range for the homoduplexes indicates the presence of a mutation or polymorphism.

In the case that the extension products happen to have overlapping retention times/elution behaviors, the DHPLC conditions can be adjusted to include a primary separation on the basis of size prior to increasing the concentration of the denaturant on the column to improve resolution. The techniques described in Huber, et al., Anal. Chem. 67:578 (1995), hereby expressly incorporated by reference in its entirety, can be adapted for use with the novel DHPLC separation approach described herein. In one embodiment, for example, the alkylated non-porous poly(styrene-divinylbenzene) DHPLC column can be used to separate the extension products on the basis of size for a time sufficient to group the various populations of extension products (i.e., the homoduplexes and heteroduplexes generated from a single independent set of primers constitute a single population of extension products) prior to separating on the basis of melting behavior.

By one approach, the extension products are applied to the column, as above, in Buffer A and a shallow linear gradient of Buffer B (e.g., 30%-50% of a solution of 0.1M TEAA pH 7.0 and 25% acetonitrile for 200-450 bp extension products) is applied so as to resolve the various populations of extension products. Then, a deeper linear gradient of Buffer B (e.g., 50%-65% of a solution of 0.1M TEAA pH 7.0 and 25% acetonitrile for 200-450 bp extension products) is applied to resolve the homoduplexes from the heteroduplexes within each individual population of extension product. In this manner, the homoduplexes and heteroduplexes from each population of extension product can be resolved despite having overlapping elution behaviors.

It should be understood that the separation based on size can be performed at virtually any temperature as long as the extension products do not denature on the column, however, the amount of denaturant in Buffer B and the type of gradient may have to be adjusted. For example, the size separation can be accomplished at 4° C.-23° C., or 23° C.-40° C., or 40′-50° C., or 50° C.-60° C. Additionally, the size separation can be accomplished while the column is being gradually equilibrated to the temperature that is going to be used for the DHPLC. It should also be understood that the size separation can be performed on the same column with the appropriate gradient (shallow for a time sufficient to separate on the basis of size followed by a deeper gradient to separate on the basis of melting behavior). Additionally, columns in series can be used to separate extension products that have overlapping retention times/elution behaviors. For example, a first DHPLC column can be used to separate on the basis of size and a second DHPLC column can be used to separate on the basis melting behavior.

Mutations or polymorphisms are easily identified using the DHPLC techniques above by comparing the elution behavior of the DNA to be screened with the elution behavior of a control DNA. As above, desirable “control” DNA or “standard” DNA includes a DNA that is wild-type or non-polymorphic for at least one loci that is screened and preferred standard DNA is wild-type or non-polymorphic for all of the loci that are being screened. Control or standard DNA can also include extension products that are homoduplexes by virtue of a mutation or polymorphism or plurality of mutations or polymorphisms. Since the elution behavior of the wild type or non-polymorphic DNA or a homozygous mutant or polymorphism, represents the elution behavior of a homoduplex, one can use DHPLC values obtained from separating these controls, such as the retention time, elution time, or amount of denaturant required to elute the homoduplex as a basis for comparison to a screened sample to identify the presence of homoduplexes. Similarly, a control DNA can be a known heteroduplex and the elution behavior values described above can be used to identify the presence of a heteroduplex in a screened sample.

Additionally, the separated extension products can be collected after passing through the DHPLC column or TTGE gel or reamplified and sequenced to verify the existence of the mutation or polymorphism. Further, the identified products can be isolated from the gel and sequenced. Sequencing can be performed using the conventional dideoxy approach (e.g., Sequenase kit) or an automated sequencer. Preferably, all possible mutant fragments are sequenced using the CEQ 2000 automated sequencer from Beckman/Coulter and the accompanying analysis software. The mutations or polymorphisms identified by sequencing can be compiled along with the respective melting behaviors and the sizes of extension products. This data can be recorded in a database so as to generate a profile for each loci.

Additionally, this profile information can be recorded with other subject-specific information, for example family or medical history, so as to generate a subject profile. By creating such databases, individual mutations can be better characterized. Mutation analysis hardware and software can also be employed to aid in the identification of mutations or polymorphisms. For example, the “ALFexpress II DNA Analysis System”, available from Amersham Pharmacia Biotech and the “Mutation Analyser 1.01”, also available from Amersham Pharmacia Biotech, can be used. Mutation Analyser automatically detects mutations in sample sequence data, generated by the ALFexpress II DNA analysis instrument. The section below describes embodiments that allow for the identification of a mutation or polymorphism at multiple loci in a plurality of genes in a single assay.

Identification of the Presence or Absence of a Mutation or Polymorphism at Multiple Loci in a Plurality of Genes in a Single Assay

The DNA separation techniques described herein can be used to rapidly identify the presence or absence of a mutation or polymorphism at multiple loci in a plurality of genes in a single assay. Accordingly, a biological sample containing DNA is obtained from a subject and the DNA is isolated by conventional means. For some applications, it may be desired to screen the RNA of a subject for the presence of a genetic disorder (e.g., a congenital disease that arises through a splicing defect). In this case, a biological sample containing RNA is obtained, the RNA is isolated, and then is converted to cDNA by methods well known to those of skill in the art. DNA from a subject or cDNA synthesized from the mRNA obtained from a subject can be easily and efficiently isolated by various techniques known in the art. Also known in the art is the ability to amplify DNA fragments from whole cells, which can also be used with the embodiments described herein. Thus, the DNA sample for use with the embodiments described herein need only be isolated in the sense that the DNA is in a form that allows for PCR amplification.

In some embodiments, genomic DNA is isolated from a biological sample by using the Amersham Pharmacia Biotech “GenomicPrep Blood DNA Isolation Kit”. The isolation procedure involves four steps: (1) cell lysis (cells are lysed using an anionic detergent in the presence of a DNA preservative, which limits the activity of endogenous and exogenous Dnases); (2) RNAse treatment (contaminating RNA is removed by treatment with RNase A); (3) protein removal (cytoplasmic and nuclear proteins are removed by salt precipitation); and (4) DNA precipitation (genomic DNA is isolated by alcohol precipitation). EXAMPLE 1 also describes an approach that was used to isolate DNA from human blood.

Once the sample DNA has been obtained, primers that flank the desired loci to be screened are designed and manufactured. Preferably, optimal primers and optimal primer concentrations are used. Desirably, the concentrations of reagents, as well as, the parameters of the thermal cycling are optimized by performing routine amplifications using control templates. Primers can be made by any conventional DNA synthesizer or are commercially available. Optimal primers desirably reduce non-specific annealing during amplification and also generate extension products that resolve reproducibly on the basis of size or melting behavior and, preferably, both. Preferably, the primers are designed to hybridize to sample DNA at regions that flank loci that can be used to diagnose a trait, such as a congenital disease (e.g., loci that have mutations or polymorphisms that indicate a human disease).

Desirably, the primers are designed to detect loci that diagnose conditions selected from the group consisting of familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis coleceral cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease. Primers can be designed to amplify any region of DNA, however, including those regions known to be associated with diseases such as alcohol dependence, obesity, and cancer. It should be understood that the embodiments described herein can be used to detect any gene, mutation, or polymorphism found in plants, virus, molds, yeast, bacteria, and animals.

Preferred primers are designed and manufactured to have a GC rich “clamp” at one end of a primer, which allows the dsDNA to denature in a “zipper-like” fashion. As one of skill will appreciate, PCR requires a “primer set”, which includes a first and a second primer, only one of which has the GC clamp so as to allow for separation of the double stranded molecule from one end only. Since the GC clamp is significantly stable, the rest of the fragment melts but does not completely separate until a point after the inflection point of the DNA, which contains the mutation or polymorphism of interest. The denaturant in the gel or on the column allows the temperature of melting to be lower and allows the inflection point of the melt to be longer in terms of temperature and, thus, the sensitivity to temperature at the inflection point is less (i.e., increment temperature=less increment melting), which increases the resolution.

Additionally, desirable primers are designed with a properly placed GC-clamp so that extension products that contain a single melting domain are produced. Preferably, the primers are selected to complement regions of introns that flank exons containing the genetic markers of interest so that polymorphisms or mutations that reside within the early portions of exons are not masked by the GC clamp. For example, it was discovered that GC clamps significantly perturb melting behavior and can prevent the detection of a polymorphism or mutation by melting behavior if the mutation or polymorphism resides too close to the GC clamp (e.g., within 40 nucleotides). By performing amplification reactions with control templates, optimal primer design and optimal concentration can be determined. The use of computer software, including, but not limited to, WinMelt or MacMelt (Bio-Rad) and Primer Premire 5.0 can aid in the creation and optimization of primers and proper positioning of the GC-clamp. Accordingly, many of the primers described herein (SEQ. ID. Nos. 1-44) are embodiments of the invention. EXAMPLE 2 further describes the design and optimization of primers that allowed for the high-throughput multiplex PCR technique described herein.

Once optimal primers are designed and selected, the DNA sample is screened using the inventive multiplex PCR technique. In some embodiments, for example, approximately 25 ng-500 ng of template DNA (preferably, 200 ng for human genomic DNA) is suspended in a buffer comprising: 10 mM Tris (pH 8.4), 50 mM KCl, 1.5 mM MgCl₂, 200 μM dNTPs, 50 pmol of each primer, and 1 unit Taq polymerase per primer set in a total volume of 50 μl. Preferably, amplification is performed under the same conditions that were used to design the primers. In some embodiments, for example, amplification is performed on a conventional thermal cycler for 30 cycles, wherein each cycle is: 1 minute @ 95° C., 58° C. for 1 minute, 72° C. for 1 minute. Final extension is performed at 72° C. for 5 minutes. When the primers have a GC clamp, it was found that conditions often favor an amplification reaction having over 40 cycles, wherein each cycle is: 35 seconds @ 95° C., 120 seconds @ 50-57° C., and 60 seconds+3 seconds/cycle @ 72° C. Thermal cyclers are available from a number of scientific suppliers and most are suitable for the embodiments described herein.

Once the PCR reaction is complete, the extension products are desirably isolated by centrifugal microfiltration using a standard PCR cleanup cartridge, for example, Qiagen's QIAquick 96 PCR Purification Kit, according to manufacture's instructions. Isolation or purification of the extension products is not necessary to practice the invention, however. The isolated extension products can then be suspended in a non-denaturing loading buffer and either loaded directly on a DHPLC column or TTGE denaturing gel. The sample can also be denatured by heating (e.g., 95° C. for 5-10 minutes) and annealed by cooling (e.g., ice for 5-10 minutes) prior to loading onto the DHPLC column or TTGE denaturing gel. The various extension products are then separated on a TTGE denaturing gel or DHPLC column on the basis of melting behavior, as described above and, after separation, the extension products can be analyzed for the presence or absence of polymorphisms or mutations. EXAMPLES 3 and 4 describe experiments that verified that multiple loci on a plurality of genes can be screened in a single assay. The section below describes a method of genetic analysis, wherein improved sensitivity of detection was obtained by adding a DNA standard to the screened DNA.

Improved Sensitivity was Obtained when a DNA Standard was Mixed with the Screened DNA

It was also discovered that greater sensitivity in the inventive multiplex PCR reactions described herein can be obtained by mixing a DNA standard with the DNA to be tested prior to conducting amplification or after amplification but prior to separation on the basis of melting behavior. Desired DNA standards include, but are not limited to, DNA that is wild-type for at least one of the traits that are being screened and preferred DNA standards include, but are not limited to, DNA that is wild-type for all of the traits that are being screened. DNA standards can also be mutant or polymorphic DNA. In some embodiments, particularly when the control DNA is added after amplification, the DNA standard is an extension product generated from a wild-type genomic DNA or a mutant genomic DNA.

By one approach, the DNA from the subject to be screened and the DNA standard are pooled and then the amplification reaction, as described above, is performed. Accordingly, optimal primers are designed and selected and approximately 25 ng-500 ng of template DNA (preferably, 200 ng for human genomic DNA) is suspended in a buffer comprising: 10 mM Tris (pH 8.4), 50 mM KCl, 1.5 mM MgCl₂, 200 μM dNTPs, 50 pmol of each primer, and 1 unit Taq polymerase per primer set in a total volume of 50%l. Preferably, amplification is performed under the same conditions that were used to design the primers. In some embodiments, amplification is performed on a conventional thermal cycler for 30 cycles, wherein each cycle is: 1 minute @ 95° C., 58° C. for 1 minute, 72° C. for 1 minute. Final extension is performed at 72° C. for 5 minutes. When the primers have a GC clamp, however, conditions often favor an amplification reaction having over 40 cycles, wherein each cycle is: 35 seconds @ 95° C., 120 seconds @ 50-57° C., and 60 seconds+3 seconds/cycle @ 72° C.

If the subject being tested has at least one disorder that is detected by the assay then two populations of extension products are generated, a first population that corresponds to the standard DNA and a second population that corresponds to the subject's DNA having at least one mutation or polymorphism. The pool of extension products are desirably isolated from the amplification reactants, as above, and are suspended in a non-denaturing loading buffer. Preferably, the extension products are then denatured by heat (e.g., 95° C. for 5 minutes), and are allowed to anneal by cooling (e.g., ice for 5 minutes) prior to loading on the TTGE denaturing gel or DHPLC column. In this manner, the formation of heteroduplexes will be favored if the subject has a mutation or polymorphism because the two populations of extension products are not perfectly complementary. However, the isolation and denaturing/annealing steps are not necessary for some embodiments.

By another approach, the DNA standard is added to the extension products generated from the tested subject's DNA after the amplification reaction. As above, the pooled DNA sample is preferably denatured by heat (e.g., 95° C. for 5 minutes), and allowed to anneal by cooling (e.g., ice for 5 minutes). This second approach also produces heteroduplexes if the extension product and the DNA standard are not perfectly complementary.

Next, the TTGE denaturing gel or DHPLC column is loaded and the extension products are separated on the basis of melting behavior, as described above. Since heteroduplexes are less stable than homoduplexes and have a lower melting temperature, the presence or absence of a mutation or polymorphism in the tested DNA sample is easily determined. By comparing the migration behavior or elution behavior of the extension products generated from the screened DNA with the migration behavior of the DNA standard, a user can rapidly determine the presence or absence of a mutation or polymorphism (e.g., two additional bands that correspond to the single extension product will appear on the gel when a mutation or polymorphism is present in the tested DNA or a population of extension products will elute from the DHPLC column earlier than homoduplex controls or the majority of homoduplexes present in the sample). The section below describes a method of genetic analysis, wherein improved efficiency and sensitivity of detection was obtained by screening multiple DNA samples in the same assay.

Improved sensitivity was obtained when multiple DNA samples were screened in the same assay It was also discovered that an improved sensitivity of detection and increased throughput could be obtained by mixing DNA from a plurality of subjects prior to amplification. Because the frequency of mutations or polymorphisms for most disorders are very low in the population, most of the extension products generated correspond to wild-type or non-polymorphic DNA. Accordingly, most of the DNA in a reaction comprising DNA from a plurality of subjects behave similar to a DNA standard. That is, the predominant structure formed upon annealing after denaturation is a homoduplex, which can be rapidly distinguished from any heteroduplex that would appear if a subject were to have a mutation or polymorphism. Although the reaction is “dirty” from the perspective that the identity of each subject's DNA is not known initially, the identity of any polymorphic or mutant DNA can be determined through a process of elimination. For example, by repeating the analysis with smaller and smaller pools of samples, one can identify the individual(s) in the pool that have the mutation or polymorphism. Additionally, DNA standards can be used, as described above, to facilitate identification of the individual(s) having the mutation or polymorphism.

By one approach, DNA from a plurality of subjects to be tested is obtained by conventional methods, pooled, and hybridized with the desired nucleic acid primers. Accordingly, optimal primers are designed and selected and approximately 25 ng-500 ng of template DNA (preferably, 200 ng for human genomic DNA) is suspended in a buffer comprising: 10 mM Tris (pH 8.4), 50 mM KCl, 1.5 mM MgCl₂, 200 μM dNTPs, 50 pmol of each primer, and 1 unit Taq polymerase per primer set in a total volume of 50 μl. Preferably, amplification is performed under the same conditions that were used to design the primers. In some embodiments, amplification is performed on a conventional thermal cycler for 30 cycles, wherein each cycle is: 1 minute @ 95° C., 58° C. for 1 minute, 72° C. for 1 minute. Final extension is performed at 72° C. for 5 minutes. When the primers have a GC clamp, however, conditions often favor an amplification reaction having over 40 cycles, wherein each cycle is: 35 seconds @ 95° C., 120 seconds @ 50-57° C., and 60 seconds+3 seconds/cycle @ 72° C.

The pool of extension products are preferably isolated from the amplification reactants, as above, and are suspended in a non-denaturing loading buffer. Preferably, the extension products are then denatured by heat (e.g., 95° C. for 5 minutes), and are allowed to anneal by cooling (e.g., ice for 5 minutes). In this manner, the formation of heteroduplexes will be favored if the subject has a mutation or polymorphism because the two types of extension products are not perfectly complementary. Again, the isolation and denaturing/annealing steps are not performed in some embodiments.

Next, the TTGE denaturing gel or DHPLC column is loaded and the extension products are separated on the basis of melting behavior, as described above. When one of the subjects being tested has at least one trait that is detected by the screen, heteroduplexes are detected on the gel or eluting from the DHPLC column. The assay can be then repeated with smaller pools of samples and assays with a DNA standard can be conducted with individual samples to confirm the identity of the subject having the mutation or polymorphism. EXAMPLE 5 describes an experiment that verified that an improved sensitivity can be obtained by mixing a plurality of DNA samples. EXAMPLE 6 describes an experiment that verified that multiple genes and multiple loci therein can be screened in a plurality of subjects, in a single assay. EXAMPLE 7 describes the screening of multiple genes and multiple loci therein, in a plurality of subjects, in a single assay using a DHPLC approach. The example below describes an approach that was used to isolate DNA from human blood.

### EXAMPLE 1

A sample of blood was obtained from a subject to be tested by phlebotomy. A portion of the sample (e.g., approximately 1.0 ml) was added to approximately three times the sample volume or 3.0 ml of a lysis solution (10 mM KHCO₃, 155 mM NH₄Cl, 0.1 mM EDTA) and was mixed gently. The lysis solution and blood were allowed to react for approximately five minutes. Next, the sample was centrifuged (x500 g) for approximately 2 minutes and the supernatant was removed. Some of the supernatant was left (e.g., on the Corporation. Information regarding mutations or polymorphisms was obtained from The Human Gene Mutation Database.

One of the primers in each primer set contained a GC-clamp. It was discovered that the addition of a GC-clamp significantly altered the melting profile of the DNA extension product. Further, proper positioning of the GC-clamp served to level the melting profile. It was desired to position the GC-clamp so that a single melting domain across the fragment was created. Proper positioning of the GC-clamp was oftentimes needed to prevent the GC-clamp from masking the presence of a mutation or polymorphism (e.g., if the mutation or polymorphism is too close to the GC-clamp). Software was also used to optimize primer design. For example, many primers were designed with the aid of Primer Premiere 4.0 and 5.0 and appropriate positioning of the GC-clamps was determined using WinMelt software from BioRad. To maintain sensitivity of the test, the primers were designed to anneal at a minimum of 40 base pairs either upstream or downstream of the nearest known mutation in the intronic region of the genes.

Although multiplex PCR can be technically difficult when using the quantity of primers described herein, it was discovered that almost all of the PCR artifacts disappeared when salt concentration, temperature, primer selection, and primer concentration were carefully optimized. Optimization was determined for each primer set alone and in combination with other primer sets. Optimization experiments were conducted using Master Mix from Qiagen and a Thermocyler from MJ Research. The conditions for thermal cycling were 5 minutes @ 95° C. for the initial denaturation, then 30 cycles of: 30 seconds @ 94° C., 45 seconds @ 48-68° C., and 1 minute @ 72° C. A final extension was performed at 72° C. for 10 minutes.

In addition to primer compatibility, primers were selected to facilitate identification of extension products by electrophoresis. To optimize primer design in this regard, separate PCR reactions were conducted for each individual set of primers and the extension products were separated by the inventive DNA separation technique, described above. Identical parameters were maintained for each assay and the migration behavior for each extension product was analyzed (e.g., compared to a standard to determine a R_{f} value for each fragment). An R_{f} value is a unit less value that characterizes a fragment's mobility relative to a standard under set conditions. In many primer optimization experiments, for example, the generated extension products were compared to a standard extension product obtained from amplification of the first exon of the PAH (phenylalanine hydroxylase) gene. A measurement of the distance of migration of each band in comparison to the distance of migration of the first exon of PAH was recorded and the R_{f} value was calculated according to the following:

$${R}_{f} = \frac{( migration distance of fragment ) cm}{( migration distance of PAH exon 1 ) cm}$$

By conducting these experiments, it was verified that the selected primers (SEQ. ID. Nos.1-44) did not produce extension products that overlapped on the gel. Optimal primer selection was obtained when optimal PCR parameters were maintained and the extension products produced dissimilar R_{f} values. Finally, the multiplex PCR was tested with all sets of primers and it was verified that few artifacts were created during amplification. Embodiments of the invention include the primers provided in the sequence listing. (SEQ. ID. Nos. 1-44). The example below describes an experiment that verified that the embodiments described herein effectively screen multiple loci present on a plurality of genes in a single assay.

### EXAMPLE 3

Two independent PCR reactions were conducted to demonstrate that multiple loci on a plurality of genes can be screened in a single assay using an embodiment of the invention. In a first reaction, seven different loci from four different genes were screened and, in the second reaction, eight different loci from four different genes were screened. The primers used in each multiplex reaction are provided in TABLE 1.

TABLE 1*

| Multiplex #1 | Multiplex #2 |
|---|---|
| Factor VIII 4 | (SEQ. ID. Nos. 7 and 25) CFTR 23 |
| (SEQ. ID. Nos. 3 and 21) | (SEQ. ID. Nos. 9 and 27) CFTR 18 |
| Factor VIII 11 | (SEQ. ID. Nos. 10 and 28) Factor VIII 11 |
| (SEQ. ID. Nos. 2 and 20) | Factor VIII 3 (SEQ. ID. Nos. 6 and 24) |
| Factor VIII 24 | CFTR 24 (SEQ. ID. Nos. 37 and 38) |
| (SEQ. ID. Nos. 9 and 27) | (SEQ. ID. Nos. 4 and 22) GBA 4 |
| PAH 9 | GALT 9 (SEQ. ID. Nos. 17 and 35) |
| (SEQ. ID. Nos. 18 and 36) | GBA 3 (SEQ. ID. Nos. 13 and 31) |
| GBA 6 | |
| (SEQ. ID. Nos. 15 and 33) | |
| Factor VIII 1 | |
| (SEQ. ID. Nos. 14 and 32) | |
| GALT 9 | |
| (SEQ. ID. Nos. 17 and 35) | |

*Primers are stored in a 50 μM storage stock and a 12.5 μM working stock.
Abbreviations are:
Phenyl alanine hydroxylase (PAH), Glucocerebrosidase (GBA), Galactose-1-phosphate uridyl transferase (GALT), and cystic fibrosis transmembrane reductase (CFTR).
The numbers following the abbreviations represent the exons probed.

The amplification was carried out in 25 μl reactions using a 2X Hot Start Master Mix, which contains Hot Start Taq DNA Polymerase, and a final concentration of 1.5 mM MgCl₂ and 200 μM of each dNTP (commercially available from Qiagen). In each reaction, 12.5 μl of Hot Start Master Mix was mixed with 1 μl of genomic DNA (approximately 200 ng genomic DNA), which was purified from blood using a commercially available blood purification kit (Pharmacia or Amersham). Primers were then added to the mixture (0.5 μM final concentration of each primer). Then, ddH₂O was added to bring the final volume to 25 μl.

Thermal cycling for the Multiplex #1 reaction was performed using the following parameters: 15 minutes @ 95° C. for 1 cycle; 30 seconds @ 94° C., 1 minute @ 53° C., 1 minute and 30 seconds ® 72° C. for 35 cycles; and 10 minutes @ 72° C. for 1 cycle. Thermal cycling for the Multiplex #2 reaction was performed using the following parameters: 15 minutes @ 95° C. for 1 cycle; 30 seconds @ 94° C., 1 minute @ 49° C., 1 minute and 30 seconds @ 72° C. for 35 cycles; and 10 minutes @ 72° C. for 1 cycle.

After the amplification was finished, approximately 5 μl of each PCR product was mixed with 5 μl of non-denaturing gel loading dye (70% glycerol, 0.05% bromophenol blue, 0.05% xylene cyanol, 2 mM EDTA). The DNA in the two reactions was then separated on the basis of melting behavior on separate denaturing gels. Each gel was a 16×16 cm, 1 mm thick, 7M urea, 8% acrylamide/bis (37.5:1) gel composed in 1.25× TAE (50 mM Tris, 25 mM acetic acid, 1.25 mM EDTA). Separation was conducted for 4 hours at 150 V on the Dcode system (BioRad) and the temperature ranged from 51° C. to 63° C. with a temperature ramp rate of 3° C./hour. Subsequently, the gels were stained in 1 μg/ml ethidium bromide in 1.25× TAE for 3 minutes and destained in 1.25× TAE buffer for 20 minutes. The gels were then photographed using the Gel Doc 1000 system from BioRad.

The primers in TABLE 1 were selected and manufactured because they produced extension products with very different R_{f} values and the extension products were clearly resolved by separation on the basis of melting behavior. Although some bands were more visible than others on the gel, seven distinct bands were observed on the gel loaded with extension products generated from the Multiplex 1 reaction and eight distinct bands were observed on the gel loaded with extension products generated from the Multiplex 2 reaction. These results verified that the described method effectively screened multiple loci on a plurality of genes in a single assay. The example below describes another experiment that verified that the embodiments described herein can be used to effectively screen multiple loci present on a plurality of genes in a single assay.

### EXAMPLE 4

Experiments were conducted to differentiate extension products generated from wild-type DNA and extension products generated from mutant DNA. Samples of genomic DNA that had been previously identified to contain mutations or polymorphisms were purchased from Coriell Cell Repositories. The mutation or polymorphism that was analyzed in this experiment was the delta-F508 mutation of the CFTR gene. This mutation is a 4 bp deletion in exon 10 of the CFTR gene. Other loci analyzed in these experiments included the Fragile X gene, exon 17; Fragile X gene, exon 3; Factor VIII gene exon 2; and the Factor VIII gene, exon 7. Both the known mutant and a control wild-type for CFTR exon 10 were amplified within a multiplex reaction and individually.

PCR amplification was conducted as described in EXAMPLE 3; however, 0.25 μM (final concentration) of each primer was used. The primers used in these experiments were CFTR 10 (SEQ. ID. Nos. 1 and 19), FragX 17 (SEQ. ID. Nos. 12 and 30), FragX 3 (SEQ. ID. Nos.11 and 29), Factor VIII 7 (SEQ. ID. Nos. 8 and 26) and Factor VIII 2 (SEQ. ID. Nos. 5 and 23). The numbers following the abbreviations represent the exons probed.

The DNA templates that were analyzed included known wild-type genomic DNA, known mutant genomic DNA, mixed wild-type genomic DNA from various subjects, and mixed wild-type and mutant genomic DNA. Approximately 200 ng of genomic DNA was added to each reaction. The mixed wild-type and mutant DNA sample had approximately 100 ng of each DNA type. Thermal cycling was carried out with a 15-minute. step at 95° C. to activate the Hot Start Polymerase, followed by 30 cycles of 30 seconds at @ 94 C, 1 minute at @ 53° C., 1 minute and 30 seconds at @ 72° C.; and 72° C. for 10 minutes.

After amplification, approximately 5 μl of the PCR product was mixed with 5 μl of non-denaturing gel loading dye (70% glycerol, 0.05% bromophenol blue, 0.05% xylene cyanol, 2 mM EDTA). The samples were then separated on a 16×16 cm, 1 mm thick, 6M urea, 6% acrylamide/bis (37.5:1) gel in 1.25× TAE (50 mM Tris, 25 mM acetic acid, 1.25 mM EDTA) for 5 hours at 130 V using the Dcode system (BioRad). The temperature ranged from 40° C. to 50° C. at a temperature ramp rate of 2° C./hour. The gels were then stained in 1 μg/ml ethidium bromide in 1.25× TAE for 3 minutes and destained in 1.25× TAE buffer for 20 minutes. The gels were then photographed using the Gel Doc 1000 system from BioRad.

The resulting gel revealed that the lane containing the extension products generated from the wild-type DNA using the CFTR10 primers had a mobility commensurate to the wild-type DNA standard, as did the extension products generated from the other primers and the wild-type DNA. That is, a single band appeared on the gel in these lanes. The lane containing the extension products generated from the template having the F508 mutant, on the other hand, showed 2 bands. One of the bands had the same mobility as the extension products generated from the wild-type or DNA standard and the other band migrated slightly faster. These two populations of bands represent the two populations of homoduplexes (i.e., wild-type/wild-type and mutant/mutant). The top band is the wild-type homoduplex and the lower band is the mutant F508 homoduplex. Similarly, the lane that contained the wild-type/mutant DNA mix exhibited two populations of extension products, one representing the wild-type homoduplex population and the other representing the mutant homoduplex. Since F508 is a 4 bp deletion it failed to form heteroduplex bands in sufficient quantity to be visible on the gel. Thus, this experiment demonstrated that the described method effectively screened multiple genes, in a single assay, and detected the presence of a polymorphism in one of the screened genes. The example below describes an experiment that demonstrated that an improved sensitivity can be obtained by mixing a plurality of DNA samples.

### EXAMPLE 5

This example describes two experiments that verified that an improved sensitivity of detection can be obtained by (1) mixing the DNA samples from a plurality of subjects prior to amplification or by (2) mixing amplification products before separation on the basis of melting behavior. In these experiments, PCR amplifications of exon 9 of the GBA gene (Glucocerebrosidase gene) were used. DNA samples known to contain a mutation in exon 9 of the GBA gene were purchased from Coriell Cell Repositories. These DNA samples contain a homozygous mutation in exon 9 of the GBA gene (the N370S mutation).

In a first experiment, single amplification of exon 9 was performed in a 25 μl reaction. A Taq PCR Master Mix (containing Taq DNA Polymerase and a final concentration of 1.5 mM MgCl₂ and 200 μM dNTPs)(Qiagen) was mixed with 0.5 μM (final concentration) of primers (SEQ. ID. Nos. 16 and 34). The template genomic DNAs analyzed in this experiment included wild-type DNA, mutant DNA, and various mixtures of wild-type and mutant DNA. For the single non-mixed reactions, approximately 200 ng of genomic DNA was used for amplification. In the mixed samples, approximately 200 ng of DNA was again used, however, the percentage of wild-type to mutant genomic DNA varied. Thermal cycling was performed according to the following parameters: 10 minutes @ 94° C.; 30 cycles of 30 seconds @ 94° C., 1 minute @ 44.5° C., and 1 minute and 30 seconds @ 72° C.; and 10 minutes @ 72° C.

In the second experiment, the amplification products were mixed prior to separation on the basis of melting behavior. Amplification of both wild-type and mutant (N370S) exon 9 of the GBA gene was performed using 25 μl reactions, as before. The Taq Master Mix obtained from Qiagen was mixed with 200 ng of genomic DNA and 0.5 μM final concentration of both primers (SEQ. ID. Nos. 16-34). PCR was carried out for 30 cycles with an annealing temperature of 56° C. for 1 minute. The denaturation and elongation steps were 94° C. for 30 seconds and 72° C. for 1 minute and 30 seconds. Final elongation was carried out at 72° C. for 10 minutes. The extension products obtained from the single amplification of wild-type GBA exon 9 was then mixed with the extension products obtained from the single amplification of the mutant GBA exon 9. Next, the pooled DNA was subjected to denaturation at 95° C. for 10 minutes and cooled on ice for 5 minutes, then heated to 65° C. for 5 minutes and cooled to 4° C. This denaturation and annealing procedure was performed to facilitate the formation of heteroduplex DNA.

Once the extension products from both experiments were in hand, approximately 5 μl of both the prior to PCR mixture and post PCR mixture were loaded on 16×16 cm, 1 mm thick gels (7M Urea/8% acrylamide (37.5:1) gel in 1.25× TAE) using the gel loading dye and the Dcode system (BioRad), described above. The DNA on the gel was then separated at 150 V for 5 hours and the temperature was uniformly raised 2° C./hour throughout the run starting at 50° C. and ending at 60° C. The gel was stained in 1 μg/ml ethidium bromide in 1.25× TAE buffer for 3 minutes and destained in buffer for minutes.

It should be noted that the GBA gene has a pseudo gene, which was co-amplified by the procedure above. An extension product generated from this psuedo gene migrated slightly faster than the extension product generated from the true expressed gene on the gel. In all lanes, the band representing the extension product generated from the psuedo gene was present. Then next fastest band on the gel was the extension product generated from the GBA exon 9 wild-type allele. The extension product generated from the mutant GBA exon 9 allele comigrated with the wild-type allele and was virtually indistinguishable on the basis of melting behavior due to the single base difference.

The heteroduplexes formed in the mixed samples were easily differentiated from the homoduplexes. The samples mixed prior to PCR showed both homoduplexes (wild-type and mutant) along with heteroduplexes, which appeared higher on the gel. Thus, by mixing samples, either prior to amplification or prior to separation on the basis of melting behavior an improved sensitivity of detection was obtained. Since homoduplex bands no longer need to be resolved to identify a mutation or polymorphism, only the heteroduplex bands need to be resolved, the throughput of diagnostic analysis was greatly improved. The example below describes experiments that verified that the embodiments taught herein can be used to effectively screen multiple genes in a plurality of subjects, in a single assay, for the presence or absence of a polymorphism or mutation.

### EXAMPLE 6

Two experiments were conducted to verify that multiple genes from a plurality of subjects can be screened in a single assay for the presence or absence of a genetic marker (e.g. a polymorphism or mutation) that is indicative of disease. These experiments also demonstrated that an improved sensitivity of detection could be obtained by mixing DNA samples either prior to generation of extension products or prior to separation on the basis of melting behavior.

In both experiments, five different extension products were generated from three different genes in a single reaction vessel. The five different extension products were generated using the following primers: Factor VIII 1 (SEQ. ID. Nos. 4 and 22); GBA 9 (SEQ. ID. Nos. 16 and 34); GBA 11 (SEQ. ID. Nos. 39 and 40); GALT 5 (SEQ. ID. Nos. 41 and 42), and GALT 8 (SEQ. ID. Nos. 43 and 44). Abbreviations are: Glucocerebrosidase (GBA) and Galactose-1-phosphate uridyl transferase (GALT). The numbers following the abbreviations represent the exons probed.

Extension products were generated for each experiment in 25 μlamplification reactions using Qiagen's 2X Hot Start Master Mix (Contains Hot Start Taq DNA Polymerase, and a final concentration of 1.5 mM MgCl₂ and 200 μM of each dNTP). To each reaction, 12.5 μl of Hot Start Master Mix was added to 1 μl of genomic DNA (approximately 200 ng genomic DNA for the mutant DNA sample and the wild-type DNA sample), which was purified from human blood using Pharmacia Amersham Blood purification kits. For the experiment in which the DNA samples from a plurality of subjects were mixed prior to generation of the extension products, approximately 100 ng of wild-type genomic DNA was mixed with approximately 100 ng of mutant N370S genomic DNA. In both experiments, primers were added to achieve a final concentration of 0.5 μM for each primer and a final volume of 25 μl was obtained by adjusting the volume with ddH₂O.

Thermal cycling for both experiments was performed using the following parameters: 15 minutes ® 95° C. for 1 cycle; 30 seconds @ 94° C., one minute @ 57° C., and one minute 30 seconds @ 72° C. for 35 cycles; and 10 minutes @ 72° C. for 1 cycle. After amplification, the extension products generated from the wild-type and mutant templates (the un-mixed samples) were separated from the PCR reactants using a PCR Clean Up kit (Qaigen). Then, approximately 10 μL of the wild-type and mutant DNA were removed from each tube and gently mixed in a single reaction vessel. This preparation was then denatured at 95° C. for 1 minute and rapidly cooled to 4° C. for 5 minutes. Finally, the preparation was brought to 65° C. for an additional 1.5 minutes. The extension products generated from the mixed sample (wild-type DNA and mutant DNA mixed prior to amplification) were stored until loaded onto a denaturing gel.

Next, approximately 10 μl of the unmixed sample was combined with 10 μl of loading dye and approximately 5 μl of the mixed sample was combined with 5 μl of loading dye. The loading dye was composed of 70% glycerol, 0.05% bromophenol blue, 0.05% xylene cyanol, and 2 mM EDTA). The samples in loading dye were then loaded on separate 16×16 cm, 1 mm thick, 7M urea, 8% acrylamide/bis (37.5:1) gels in 1.25× TAE (50 mM Tris, 25 mM acetic acid, 1.25 mM EDTA). The DNA was separated on the basis of melting behavior for 5 hours at 150 V on the Dcode system (BioRad). The temperature ranged from 56° C. to 68° C. at a temperature ramp rate of 2° C./hr. The gels were then stained in 1 μg/ml ethidium bromide in 1.25× TAE for 3 minutes and destained in 1.25× TAE buffer for 20 minutes. The gels were photographed using the Gel Doc 1000 system (BioRad).

In all lanes of the gel, 5 extension products generated from three different genes were visible in the following order from top to bottom: Factor VIII 1, GBA 9, GBA 11, GALT 8, and GALT 5. Two different extension products were generated from the GBA 9 primers, as described above. The less intense band below the homoduplex bands corresponded to an extension product generated from the pseudogene. In the lanes loaded with extension products generated from only the wild-type or mutant DNA template, it was difficult to distinguish the wild type homoduplex from the N370S mutant homoduplex. In the lane loaded with the extension products generated from the mixed DNA templates (wild-type and mutant DNA mixed prior to amplification) and the lane loaded with extension products (generated from wild type and mutant DNA separately) that were mixed after amplification, heteroduplex bands were easily visualized. These experiments verified that multiple genes can be screened in a plurality of individuals in a single assay and that a single nucleotide mutation or polymorphism can be detected. Further, these experiments demonstrate that screening a plurality of DNA samples in a single reaction vessel or adding a control DNA before or after amplification greatly improves the sensitivity of detection. By practicing the methods taught in this example, the throughput of diagnostic screening can be drastically improved and the cost of identifying genetic traits can be significantly reduced. The example below describes approaches to screen multiple genes in a plurality of subjects, in a single assay, for the presence or absence of a polymorphism or mutation using DHPLC.

### EXAMPLE 7

Multiple genes in a plurality of subjects, in a single assay, can be screened for the presence or absence of a polymorphism or mutation using a DHPLC separation approach.

For example, five different extension products can be generated using the following primers: Factor VIII 1 (SEQ. ID. Nos. 4 and 22); GBA 9 (SEQ. ID. Nos. 16 and 34); GBA 11 (SEQ. ID. Nos. 39 and 40); GALT 5 (SEQ. ID. Nos. 41 and 42), and GALT 8 (SEQ. ID. Nos. 43 and 44). Abbreviations are: Glucocerebrosidase (GBA) and Galactose -1-phosphate uridyl transferase (GALT). The numbers following the abbreviations represent the exons probed. The extension products can be generated in 251 μl amplification reactions using Qiagen's 2X Hot Start Master Mix (Contains Hot Start Taq DNA Polymerase, and a final concentration of 1.5 mM MgCl₂ and 200 μM of each dNTP).

To each reaction, 12.5 μl of Hot Start Master Mix is added to 1 μl of genomic DNA (approximately 200 ng genomic DNA for the mutant DNA sample and the wild-type DNA sample), which is purified from human blood using Pharmacia Amersham Blood purification kits. By another approach, the DNA samples from a plurality of subjects can be mixed prior to generation of the extension products. In this case, approximately 100 ng of wild-type genomic DNA is mixed with approximately 100 ng of mutant N370S genomic DNA. Primers are added to achieve a final concentration of 0.5 μM for each primer and a final volume of 25 μl is obtained by adjusting the volume with ddH₂O.

Thermal cycling is performed using the following parameters: 15 minutes @ 95° C. for 1 cycle; 30 seconds @ 94° C., one minute @ 57° C., and one minute 30 seconds @ 72° C. for 35 cycles; and 10 minutes @ 72° C. for 1 cycle. After amplification, the extension products generated from the wild-type and mutant templates (if un-mixed samples) are separated from the PCR reactants using a PCR Clean Up kit (Qiagen). Then, approximately 10 μL of the wild-type and mutant DNA are removed from each tube and gently mixed in a single reaction vessel. This preparation is then denatured at 95° C. for 1 minute and rapidly cooled to 4° C. for 5 minutes. Finally, the preparation is brought to 65° C. for an additional 1.5 minutes. The extension products generated from the mixed sample (wild-type DNA and mutant DNA mixed prior to amplification) can be stored until loaded onto a DHPLC column.

Next, the extension products are loaded on to a 50×4.6 mm ion pair reverse phase HPLC column that is equilibrated in degassed Buffer A (0.1 M triethylamine acetate (TEAA) pH 7.0) at 56° C. A linear gradient of 40%-50% of degassed Buffer B (0.1 M triethylamine acetate (TEAA) pH 7.0 and 25% acetonitrile) is then performed over 2.5 minutes at a flow rate of 0.9 ml/min at 56° C., followed by a linear gradient of 50%-55.3% Buffer B over 0.5 minutes, and finally a linear gradient of 55.3%-61% Buffer B over 4 minutes. U.V. absorption is monitored at 260 nm, recorded and plotted against retention time.

When the loaded sample is un-mixed extension products, the extension products generated from only the wild-type or mutant DNA template, it is difficult to distinguish the wild type homoduplex from the N370S mutant homoduplex. When the loaded sample is the mixed extension products, the extension products generated from the mixed DNA templates (wild-type and mutant DNA mixed prior to amplification), or the extension products (generated from wild type and mutant DNA separately) that were mixed after amplification, heteroduplex elution behavior is detected. By practicing the methods taught in this example, the throughput of diagnostic screening can be drastically improved and the cost of identifying genetic traits can be significantly reduced.

Although the invention has been described with reference to embodiments and examples, it should be understood that various modifications can be made without departing from the spirit of the invention. Accordingly, the invention is limited only by the following claims.

What is claimed is:

1. A method of identifying the presence or absence of a plurality of genetic markers in a subject comprising:
providing a DNA sample from said subject;
providing a plurality of nucleic acid primer sets that hybridize to said DNA at regions that flank said plurality of genetic markers, wherein each primer set has a first and a second primer and, wherein said plurality of genetic markers exist on a plurality of genes;
contacting said DNA and said plurality of nucleic acid primer sets in a single reaction vessel;
generating, in said single reaction vessel, a plurality of extension products that comprise regions of DNA that include the location of said plurality of genetic markers;
separating said plurality of extension products on the basis of melting behavior; and
identifying the presence or absence of said plurality of genetic markers in said subject by analyzing the melting behavior of said plurality of extension products.

2. The method of claim 1, wherein said subject is selected from the group consisting of a plant, virus, bacteria, mold, yeast, animal, and human.

3. The method of claim 1, wherein either said first or said second primer comprise a GC clamp.

4. The method of claim 1, wherein either said first or said second primer hybridize to a sequence within an intron.

5. The method of claim 1, wherein at least one of said plurality of genetic markers is indicative of a disease selected from the group consisting of familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis coleceral cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease.

6. The method of claim 1, wherein said plurality of primer sets consist of at least 3 primer sets.

7. The method of claim 1, wherein said plurality of primer sets consist of at least 4 primer sets.

8. The method of claim 1, wherein said plurality of primer sets consist of at least 5 primer sets.

9. The method of claim 1, wherein said plurality of primer sets consist of at least 6 primer sets.

10. The method of claim 1, wherein said plurality of primer sets consist of at least 7 primer sets.

11. The method of claim 1, wherein said plurality of genes consist of at least 2 genes.

12. The method of claim 1, wherein said plurality of genes consist of at least 3 genes.

13. The method of claim 1, wherein said plurality of genes consist of at least 4 genes.

14. The method of claim 1, wherein said plurality of genes consist of at least 5 genes.

15. The method of claim 1, wherein said plurality of genes consist of at least 6 genes.

16. The method of claim 1, wherein said plurality of genes consist of at least 7 genes.

17. The method of claim 1, wherein said extension products are generated by Polymerase Chain Reaction.

18. The method of claim 1, further comprising adding a control DNA.

19. The method of claim 1, wherein the separation on the basis of melting behavior comprises temperature gradient gel electrophoresis (TTGE).

20. The method of claim 1, wherein the separation on the basis of melting behavior comprises denaturing high performance liquid chromatography (DHPLC).

21. The method of claim 20, wherein said DHPLC comprises an ion-pair reverse phase column.

22. The method of claim 1, further comprising a separation on the basis of size.

23. A method of identifying the presence or absence of a plurality of genetic markers in a plurality of subjects comprising:
providing a DNA sample from said plurality of subjects;
providing a plurality of nucleic acid primer sets that hybridize to said DNA at regions that flank said plurality of genetic markers, wherein each primer set has a first and a second primer and, wherein said plurality of genetic markers exist on a plurality of genes;
contacting said DNA and said plurality of nucleic acid primer sets in a single reaction vessel;
generating, in said single reaction vessel, a plurality of extension products that comprise regions of DNA that include the location of said plurality of genetic markers;
separating said plurality of extension products on the basis of melting behavior; and
identifying the presence or absence of said plurality of genetic markers in said plurality of subjects by analyzing the melting behavior of said plurality of extension products.

24. The method of claim 23, wherein said subject is selected from the group consisting of a plant, virus, bacteria, mold, yeast, animal, and human.

25. The method of claim 23, wherein either said first or said second primer comprise a GC clamp.

26. The method of claim 23, wherein either said first or said second primer hybridize to a sequence within an intron.

27. The method of claim 23, wherein at least one of said plurality of genetic markers is indicative of a disease selected from the group consisting of familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis coleceral cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease.

28. The method of claim 23, wherein said plurality of subjects consist of at least 2 subjects.

29. The method of claim 23, wherein said plurality of subjects consist of at least 3 subjects.

30. The method of claim 23, wherein said plurality of subjects consist of at least 4 subjects.

31. The method of claim 23, wherein said plurality of subjects consist of at least subjects.

32. The method of claim 23, wherein said plurality of subjects consist of at least 6 subjects.

33. The method of claim 23, wherein said plurality of subjects consist of at least 7 subjects.

34. The method of claim 23, wherein said plurality of primer sets consist of at least 3 primer sets.

35. The method of claim 23, wherein said plurality of primer sets consist of at least 4 primer sets.

36. The method of claim 23, wherein said plurality of primer sets consist of at least 5 primer sets.

37. The method of claim 23, wherein said plurality of primer sets consist of at least 6 primer sets.

38. The method of claim 23, wherein said plurality of primer sets consist of at least 7 primer sets.

39. The method of claim 23, wherein said plurality of genes consist of at least 2 genes.

40. The method of claim 23, wherein said plurality of genes consist of at least 3 genes.

41. The method of claim 23, wherein said plurality of genes consist of at least 4 genes.

42. The method of claim 23, wherein said plurality of genes consist of at least 5 genes.

43. The method of claim 23, wherein said plurality of genes consist of at least 6 genes.

44. The method of claim 23, wherein said plurality of genes consist of at least 7 genes.

45. The method of claim 23, wherein said extension products are generated by Polymerase Chain Reaction.

46. The method of claim 23, wherein the separation on the basis of melting behavior comprises temperature gradient gel electrophoresis (TTGE).

47. The method of claim 23, wherein the separation on the basis of melting behavior comprises denaturing high performance liquid chromatography (DHPLC).

48. The method of claim 47, wherein said DHPLC comprises an ion-pair reverse phase column.

49. The method of claim 23, further comprising a separation on the basis of size.

50. A method of identifying the presence or absence of a mutation or polymorphism in a subject comprising:
providing a DNA sample from said subject;
generating a population of extension products from said sample, wherein said extension products comprise a region of said DNA that corresponds to the location of said mutation or polymorphism;
providing at least one control DNA, wherein said control DNA lacks said mutation or polymorphism;
contacting said control DNA and said population of extension products in a single reaction vessel thereby forming a mixed DNA sample;
heating said mixed DNA sample to a temperature sufficient to denature said control DNA and said DNA sample;
cooling said mixed DNA sample to a temperature sufficient to anneal said control DNA and said DNA sample;
separating said mixed DNA sample on the basis of melting behavior; and
identifying the presence or absence of said mutation or polymorphism by analyzing the melting behavior of said mixed DNA sample.

51. The method of claim 50, wherein said control DNA is DNA obtained from a second subject and, wherein, the presence or absence of said mutation or polymorphism is not known.

52. The method of claim 50, wherein the separation on the basis of melting behavior comprises temperature gradient gel electrophoresis (TTGE).

53. The method of claim 50, wherein the separation on the basis of melting behavior comprises denaturing high performance liquid chromatography (DHPLC).

54. The method of claim 53, wherein said DHPLC comprises an ion-pair reverse phase column.

55. The method of claim 50, further comprising a separation on the basis of size.

56. An isolated or purified nucleic acid consisting of a sequence selected from the group consisting of SEQ. ID. Nos. 1-44.

57. A kit comprising an isolated or purified nucleic acid consisting of the sequence selected from the group consisting of SEQ. ID. Nos. 1-44.

58. The kit of claim 45, further comprising a control DNA.

59. A kit for performing amplification on a plurality of discrete genes of a subject, comprising:
a mixture of at least 3 primer sets, each of said primer set adapted to amplify a DNA associated with a different genetic trait of said subject.

60. A reaction vessel comprising:
a DNA sample obtained from a subject; and
a plurality of nucleic acid primer sets that hybridize to said DNA sample at regions that flank a plurality of genetic markers, wherein said plurality of genetic markers exist on a plurality of genes.

61. The reaction vessel of claim 48, wherein said plurality of nucleic acid primers comprises at least one nucleic acid primer consisting of a sequence selected from the group consisting of SEQ. ID. Nos. 1-44.

62. A reaction vessel comprising:
a plurality of DNA samples obtained from a plurality of subjects; and
a plurality of nucleic acid primer sets that hybridize to said plurality of DNA samples at regions that flank a plurality of genetic markers, wherein said plurality of genetic markers exist on a plurality of genes.

63. The reaction vessel of claim 50, wherein said plurality of nucleic acid primers comprises at least one nucleic acid primer consisting of a sequence selected from the group consisting of SEQ. ID. Nos. 1-44.

64. A gel having lanes and adapted to separate different DNAs comprising:
a plurality of extension products, in a single lane of said gel, wherein said plurality of extension products correspond to regions of DNA located on a plurality of genes and, wherein said regions of DNA comprise loci that indicate a genetic trait.

65. A gel having lanes and adapted to separate different DNAs comprising:
a plurality of extension products, in a single lane of said gel, wherein said plurality of extension products correspond to regions of DNA located on a plurality of genes in a plurality of subjects and, wherein said regions of DNA comprise loci that indicate a genetic trait.

66. A denaturing high pressure liquid chromatography column adapted to separate different DNAs comprising:
a plurality of extension products, wherein said plurality of extension products correspond to regions of DNA located on a plurality of genes and, wherein said regions of DNA comprise loci that indicate a genetic trait.

67. A gel having lanes and adapted to separate different DNAs comprising:
a plurality of extension products, in a single lane of said gel, wherein said plurality of extension products correspond to regions of DNA located on a plurality of genes in a plurality of subjects and, wherein said regions of DNA comprise loci that indicate a genetic trait.


SEQUENCE LISTING (44 sequences)

SEQ ID NO: 1 - DNA, 58 bp, Artificial Sequence
  Diagnostic Oligonucleotide
cgcccgccgcgccccgcgcccgccccgccgcccccgcccgggatgataattggaggca

SEQ ID NO: 2 - DNA, 20 bp, Artificial Sequence
  Diagnostic Oligonucleotide
taggagaagtgtgaataaag

SEQ ID NO: 3 - DNA, 60 bp, Artificial Sequence
  Diagnostic Oligonucleotide
cgcccgccgcgccccgcgcccgccccgccgcccccgcccgctgttctgtgatattatgtg

SEQ ID NO: 4 - DNA, 61 bp, Artificial Sequence
  Diagnostic Oligonucleotide
cgcccgccgcgccccgcgcccgccccgccgcccccgcccgtttgcttctccagttgaacat

SEQ ID NO: 5 - DNA, 67 bp, Artificial Sequence
  Diagnostic Oligonucleotide
cgcccgccgcgccccgcgcccgccccgccgcccccgcccgttgaagtgtccaccaaaatgaacgact

SEQ ID NO: 6 - DNA, 61 bp, Artificial Sequence
  Diagnostic Oligonucleotide
cgcccgccgcgccccgcgcccgccccgccgcccccgcccggtactatccccaagtaacctt

SEQ ID NO: 7 - DNA, 67 bp, Artificial Sequence
  Diagnostic Oligonucleotide
cgcccgccgcgccccgcgcccgccccgccgcccccgcccgtacagtggatatagaaaggacaatttt

SEQ ID NO: 8 - DNA, 25 bp, Artificial Sequence
  Diagnostic Oligonucleotide
cagattctctacttcatagccatag

SEQ ID NO: 9 - DNA, 62 bp, Artificial Sequence
  Diagnostic Oligonucleotide
cgcccgccgcgccccgcgcccgccccgccgcccccgcccgctatttatggttttgcttgtgg

SEQ ID NO: 10 - DNA, 60 bp, Artificial Sequence
  Diagnostic Oligonucleotide
cgcccgccgcgccccgcgcccgccccgccgcccccgcccggctcagtataactgaggctg

SEQ ID NO: 11 - DNA, 59 bp, Artificial Sequence
  Diagnostic Oligonucleotide
cgcccgccgcgccccgcgcccgccccgccgcccccgcccgcaaaagttgatggcagagt

SEQ ID NO: 12 - DNA, 18 bp, Artificial Sequence
  Diagnostic Oligonucleotide
tgtcaggccaattacaga

SEQ ID NO: 13 - DNA, 58 bp, Artificial Sequence
  Diagnostic Oligonucleotide
cgcccgccgcgccccgcgcccgccccgccgcccccgcccgggggtgaggaattttgaa

SEQ ID NO: 14 - DNA, 59 bp, Artificial Sequence
  Diagnostic Oligonucleotide
cgcccgccgcgccccgcgcccgccccgccgcccccgcccgatacccttattccctgtgg

SEQ ID NO: 15 - DNA, 20 bp, Artificial Sequence
  Diagnostic Oligonucleotide
cactggttgggctagtatgt

SEQ ID NO: 16 - DNA, 58 bp, Artificial Sequence
  Diagnostic Oligonucleotide
cgcccgccgcgccccgcgcccgccccgccgcccccgcccgcccagtgttgagcctttg

SEQ ID NO: 17 - DNA, 21 bp, Artificial Sequence
  Diagnostic Oligonucleotide
gctcccagtagggtcagcatc

SEQ ID NO: 18 - DNA, 20 bp, Artificial Sequence
  Diagnostic Oligonucleotide
atggccaagtactaggttgg

SEQ ID NO: 19 - DNA, 20 bp, Artificial Sequence
  Diagnostic Oligonucleotide
ctaaccgattgaatatggag

SEQ ID NO: 20 - DNA, 60 bp, Artificial Sequence
  Diagnostic Oligonucleotide
cgcccgccgcgccccgcgcccgccccgccgcccccgcccgatactttgttacttgtctga

SEQ ID NO: 21 - DNA, 20 bp, Artificial Sequence
  Diagnostic Oligonucleotide
gttatcaagaattacaaggg

SEQ ID NO: 22 - DNA, 20 bp, Artificial Sequence
  Diagnostic Oligonucleotide
cgatcagaccctacaggaca

SEQ ID NO: 23 - DNA, 27 bp, Artificial Sequence
  Diagnostic Oligonucleotide
gatacccaatttcataaatagcattca

SEQ ID NO: 24 - DNA, 22 bp, Artificial Sequence
  Diagnostic Oligonucleotide
catagaatgacaggacaatagg

SEQ ID NO: 25 - DNA, 27 bp, Artificial Sequence
  Diagnostic Oligonucleotide
tgcttatttcatctcaatcctacgctt

SEQ ID NO: 26 - DNA, 67 bp, Artificial Sequence
  Diagnostic Oligonucleotide
cgcccgccgcgccccgcgcccgccccgccgcccccgcccgaatattcattttaaagatccaagatat

SEQ ID NO: 27 - DNA, 25 bp, Artificial Sequence
  Diagnostic Oligonucleotide
taaggggacatacactgagaatgaa

SEQ ID NO: 28 - DNA, 20 bp, Artificial Sequence
  Diagnostic Oligonucleotide
ctctgagtcagttaaacagt

SEQ ID NO: 29 - DNA, 18 bp, Artificial Sequence
  Diagnostic Oligonucleotide
atgactttatggcaggga

SEQ ID NO: 30 - DNA, 60 bp, Artificial Sequence
  Diagnostic Oligonucleotide
cgcccgccgcgccccgcgcccgccccgccgcccccgcccgtacggaaatggtataggaaa

SEQ ID NO: 31 - DNA, 19 bp, Artificial Sequence
  Diagnostic Oligonucleotide
ggtgaggggtgtaatggtt

SEQ ID NO: 32 - DNA, 21 bp, Artificial Sequence
  Diagnostic Oligonucleotide
atggctctatgtcatcttgtc

SEQ ID NO: 33 - DNA, 58 bp, Artificial Sequence
  Diagnostic Oligonucleotide
cgcccgccgcgccccgcgcccgccccgccgcccccgcccgtaggttgagggttgggac

SEQ ID NO: 34 - DNA, 21 bp, Artificial Sequence
  Diagnostic Oligonucleotide
cctcgtggtgtagagtgatgt

SEQ ID NO: 35 - DNA, 60 bp, Artificial Sequence
  Diagnostic Oligonucleotide
cgcccgccgcgccccgcgcccgccccgccgcccccgcccggagcccaggagcccagaaat

SEQ ID NO: 36 - DNA, 60 bp, Artificial Sequence
  Diagnostic Oligonucleotide
cgcccgccgcgccccgcgcccgccccgccgcccccgcccggagggccatagactatagca

SEQ ID NO: 37 - DNA, 20 bp, Artificial Sequence
  Diagnostic Oligonucleotide
tttctgtccctgctctggtc

SEQ ID NO: 38 - DNA, 60 bp, Artificial Sequence
  Diagnostic Oligonucleotide
cgcccgccgcgccccgcgcccgccccgccgcccccgcccgtcccacgagctccaattcca

SEQ ID NO: 39 - DNA, 58 bp, Artificial Sequence
  Diagnostic Oligonucleotide
cgcccgccgcgccccgcgcccgccccgccgcccccgcccgggtgaggtctgggaagtg

SEQ ID NO: 40 - DNA, 19 bp, Artificial Sequence
  Diagnostic Oligonucleotide
tgcctccttgagtatctgc

SEQ ID NO: 41 - DNA, 60 bp, Artificial Sequence
  Diagnostic Oligonucleotide
cgcccgccgcgccccgcgcccgccccgccgcccccgcccgcagccaagccctacctctcg

SEQ ID NO: 42 - DNA, 20 bp, Artificial Sequence
  Diagnostic Oligonucleotide
cttcatcaccccctccctgc

SEQ ID NO: 43 - DNA, 66 bp, Artificial Sequence
  Diagnostic Oligonucleotide
cgcccgccgcgccccgcgcccgccccgccgcccccgcccgcttgatgacttcctatccattctgtc

SEQ ID NO: 44 - DNA, 21 bp, Artificial Sequence
  Diagnostic Oligonucleotide
aacctccatccagtgcctagc